(19) United States
(12) Patent Application Publication
(10) Pub. No.: US 2006/0269611 A1
(43) Pub. Date: Nov. 30, 2006

(54) Prevention and treatment of androgen-deprivation induced osteoporosis

(76) Inventors: Mitchell S. Steiner, Germantown, TN (US); Sharan Raghow, Collierville, TN (US); Karen A. Veverka, Cordova, TN (US)

Correspondence Address: Pearl Cohen Zedek Latzer, LLP;Suite 1001, 10 Rockefeller Plaza, New York, NY, 10020, US

(21) Appl. No.: 11/329,393

(22) Filed: Jan. 11, 2006

Related U.S. Application Data
(63) Continuation-in-part of application No. 10/944,465, filed on Sep. 20, 2004.
Application No. 10/944,465 is a continuation-in-part of application No. 10/778,334, filed on Feb. 17, 2004.
Application No. 10/778,334 is a continuation-in-part of application No. 10/609,684, filed on Jul. 1, 2003.
Application No. 10/609,684 is a continuation-in-part of application No. 10/305,363, filed on Nov. 27, 2002, now Pat. No. 6,899,888.
(60) Provisional application No. 60/333,734, filed on Nov. 29, 2001.

(51) Int. Cl. A61K 9/14; A61K 31/138

(52) U.S. Cl. 424/489; 514/651

(57) ABSTRACT

This invention provides a method of treating androgen-deprivation induced osteoporosis, bone fractures or loss of bone mineral density (BMD) in a male human subject suffering from prostate cancer by administering a pharmaceutical composition comprising Toremifene and/or its analog, derivative, isomer, metabolite, pharmaceutically acceptable salt, pharmaceutical product, hydrate, N-oxide, or any combination thereof, to the subject.

### CROSS REFERENCE TO RELATED APPLICATION

This application is a continuation-in-part of U.S. application Ser. No. 10/944,465, filed Sep. 20, 2004, which is a continuation-in-part of U.S. application Ser. No. 10/778,334, filed Feb. 17, 2004, which is a continuation-in-part of U.S. application Ser. No. 10/609,684, filed Jul. 3, 2003, which is a continuation-in-part of U.S. application Ser. No. 10/305,363, filed Nov. 27, 2002, and claims priority of U.S. Provisional Application Ser. No. 60/333,734, filed Nov. 29, 2001, and is also a continuation-in-part of Ser. No. 10/778,334, filed Feb. 17, 2004, which is a continuation-in-part of U.S. application Ser. No. 10/609,684, filed Jul. 3, 2003, which is a continuation-in-part of U.S. application Ser. No. 10/305,363, filed Nov. 27, 2002, and claims priority of U.S. Provisional Application Ser. No. 60/333,734, filed Nov. 29, 2001. These applications are hereby incorporated in their entirety by reference herein.

### FIELD OF INVENTION

This invention relates to the prevention and treatment of androgen-deprivation induced conditions in men suffering from prostate cancer. More particularly, this invention relates to a method of treating, preventing, suppressing, inhibiting, or reducing the risk of developing androgen-deprivation induced osteoporosis, bone fractures, and/or loss of bone mineral density (BMD) in men suffering from prostate cancer, comprising administering to a male subject suffering from prostate cancer an anti-estrogen agent and/or its analog, derivative, isomer, metabolite, pharmaceutically acceptable salt, pharmaceutical product, hydrate, N-oxide, or any combination thereof.

### BACKGROUND OF THE INVENTION

It is well established that the bone mineral density of males decreases with age. Decreased bone mineral content and density correlates with decreased bone strength and predisposes the bone to fracture. Sex-hormones appear to play a role in bone homeostasis, with physiologic concentrations of androgens and estrogens being involved in maintaining bone health, throughout adult life. Sex hormone deprivation occurs typically results in an increase in the rate of bone remodeling, skewing the normal balance between bone resorption and formation to favor resorption, contributing to an overall loss of bone mass

Prostate cancer is one of the most frequently diagnosed noncutaneous cancers among men in the United States. One of the approaches to the treatment of prostate cancer is androgen deprivation in the subject. The male sex hormone, testosterone, stimulates the growth of cancerous prostatic cells and, therefore, is the primary fuel for the growth of prostate cancer. The goal of androgen deprivation is to decrease stimulation of cancerous prostatic cells by testosterone. Testosterone is normally produced by the testes in response to stimulation from a hormonal signal called luteinizing hormone (LH) which in turn is stimulated by luteinizing-hormone releasing hormone (LH-RH). Androgen deprivation in male subjects has been accomplished surgically, by bilateral orchidectomy, and chemically, for example, via the administration of LH-RH agonists (LHRHα) and/or antiandrogens.

Androgen deprivation in patients with micrometastatic disease has been shown to prolong survival [Messing E M, et al (1999), N Engl J Med 34, 1781-1788; Newling (2001), Urology 58(Suppl 2A), 50-55]. Moreover, androgen deprivation is being employed in numerous new clinical settings, including neoadjuvant therapy prior to radical prostatectomy, long-term adjuvant therapy for patients at high risk for recurrence following radiation or surgery, neoadjuvant therapy for radiation, and treatment of biochemical recurrence following radiation or surgery [Carroll, et al (2001), Urology 58, 14; Horwitz E M, et al (2001), Int J Radiat Oncol Biol Phy Mar 15; 49(4), 947-56]. Thus, more prostate cancer patients have become candidates for and are being treated by androgen ablation, and at an earlier time and for a prolonged period of time, than previously undertaken. Treatment lasting 10 or more years with androgen deprivation therapy is not uncommon.

Unfortunately, androgen deprivation therapy is accompanied by significant side effects, including hot flashes, gynecomastia, osteoporosis, decreased lean muscle mass, depression and other mood changes, loss of libido, and erectile dysfunction [Stege R (2000), Prostate Suppl 10, 38-42]. Consequently, complications of androgen blockade now contribute significantly to the morbidity, and in some cases the mortality, of men suffering from prostate cancer.

Given that more patients today are being treated by long-term androgen deprivation, osteoporosis has become a clinically important side effect in men suffering from prostate cancer undergoing androgen deprivation. Loss of bone mineral density (BMD) occurs in the majority of patients being treated by androgen deprivation by 6 months. New innovative approaches are urgently needed to decrease the incidence of androgen-deprivation induced osteoporosis and bone disease in men suffering from prostate cancer.

### SUMMARY OF THE INVENTION

In one embodiment, this invention provides a method of treating androgen-deprivation induced osteoporosis in a male human subject suffering from prostate cancer, said method comprising the step of administering 80 mg per day of Toremifene, or a pharmaceutically acceptable salt thereof to said subject.

In one embodiment, this invention provides method of preventing androgen-deprivation induced osteoporosis in a male human subject suffering from prostate cancer, said method comprising the step of administering 80 mg per day of Toremifene, or a pharmaceutically acceptable salt thereof to said subject.

In one embodiment, this invention provides a method of suppressing, inhibiting or reducing the risk of developing androgen-deprivation induced osteoporosis in a male human subject suffering from prostate cancer, said method comprising the step of administering 80 mg per day of Toremifene, or a pharmaceutically acceptable salt thereof to said subject.

In one embodiment, this invention provides a method of preventing androgen-deprivation induced loss of bone mineral density (BMD) in a male human subject suffering from prostate cancer, said method comprising the step of administering 80 mg per day of Toremifene, or a pharmaceutically acceptable salt thereof to said subject.

In one embodiment, this invention provides a method of preventing androgen-deprivation induced loss of bone mineral density (BMD) in a male human subject suffering from prostate cancer, said method comprising the step of administering 80 mg per day of Toremifene, or a pharmaceutically acceptable salt thereof to said subject.

In one embodiment, this invention provides a method of suppressing, inhibiting or reducing the risk of developing androgen-deprivation induced loss of bone mineral density (BMD) in a male human subject suffering from prostate cancer, said method comprising the step of administering 80 mg per day of Toremifene, or a pharmaceutically acceptable salt thereof to said subject.

In one embodiment, this invention provides a method of treating androgen-deprivation induced bone fractures in a male human subject suffering from prostate cancer, said method comprising the step of administering 80 mg per day of Toremifene, or a pharmaceutically acceptable salt thereof to said subject.

In one embodiment, this invention provides a method of preventing androgen-deprivation induced bone fractures in a male human subject suffering from prostate cancer, said method comprising the step of administering 80 mg per day of Toremifene, or a pharmaceutically acceptable salt thereof to said subject.

In another embodiment, this invention provides a method of suppressing, inhibiting or reducing the risk of developing androgen-deprivation induced bone fractures in a male human subject suffering from prostate cancer, said method comprising the step of administering 80 mg per day of Toremifene, or a pharmaceutically acceptable salt thereof to said subject.

### DETAILED DESCRIPTION OF THE INVENTION

This invention provides, in some embodiments, methods of 1) treating androgen-deprivation induced osteoporosis; 2) preventing androgen-deprivation induced osteoporosis; 3) suppressing, inhibiting or reducing the risk of developing androgen-deprivation induced osteoporosis; 4) treating androgen-deprivation induced loss of bone mineral density (BMD); 5) preventing androgen-deprivation induced loss of bone mineral density (BMD); 6) suppressing, inhibiting or reducing the risk of developing androgen-deprivation induced loss of bone mineral density (BMD); 7) treating androgen-deprivation induced bone fractures; 8) preventing androgen-deprivation induced bone fractures; 9) suppressing, inhibiting or reducing the risk of developing treating androgen-deprivation induced bone fractures in a male human subject suffering from prostate cancer by administering to the subject 80 mg per day of Toremifene and/or its analog, derivative, isomer, metabolite, pharmaceutically acceptable salt, pharmaceutical product, hydrate, N-oxide, or any combination thereof.

In males, while the natural decline in sex-hormones at maturity (direct decline in androgens as well as lower levels of estrogens derived from peripheral aromatization of androgens) is associated with the frailty of bones, this effect is more pronounced in males who have undergone androgen deprivation therapy.

As provided herein, the results demonstrate that administration of an anti-estrogen, such as, for example, Toremifene, at a daily dosage of from about 80 mg, increases bone density, as in Example 2.

Toremifene is an example of a triphenylalkylene compound described in U.S. Pat. Nos. 4,696,949 and 5,491,173 to Toivola et al., the disclosures of which are incorporated herein by reference. Formulations containing Toremifene are described, for example, in U.S. Pat. No. 5,571,534 to Jalonen et al. and in U.S. Pat. No. 5,605,700 to DeGregorio et al., the disclosures of which are incorporated herein by reference.

In one embodiment the methods of this invention are directed to Toremifene treatment, prevention, suppression, inhibition or reduction of the risk of developing androgen-deprivation induced osteoporosis and/or loss of BMD and/or a Toremifene analog, derivative, isomer, metabolite, pharmaceutically acceptable salt, pharmaceutical product, hydrate, N-oxide, or any combination thereof.

As exemplified herein, unexpectedly, a dose of 80 mg/day in humans was particularly effective in increasing bone density. In one embodiment, the dosage is administered over a prolonged period of time. In one embodiment, the treatment is provided for 1 month, or in another embodiment, for 1-6 months, or in another embodiment, for 1-12 months, or in another embodiment, for at least one year, or in another embodiment, for the duration of androgen deprivation therapy, by chemical means. In another embodiment, the treatment is continuous, or in another embodiment, the treatment is cyclic, with specified periods of treatment and lack of treatment. In another embodiment, treatment is continued and discontinued as a function of bone density or bone mineral loss, such that the subject is evaluated at specified periods, and the administration regimen is tailored to individual responses to treatment.

The present invention provides a method of treating androgen-deprivation induced osteoporosis in a male human subject suffering from prostate cancer, the method comprising the step of administering to said subject 80 mg/day of Toremifene and/or its analog, derivative, isomer, metabolite, pharmaceutically acceptable salt, pharmaceutical product, hydrate, N-oxide, or any combination thereof.

In another embodiment, the invention provides a method of preventing androgen-deprivation induced osteoporosis in a male human subject suffering from prostate cancer, comprising the step of administering to said subject 80 mg per day of Toremifene and/or its analog, derivative, isomer, metabolite, pharmaceutically acceptable salt, pharmaceutical product, hydrate, N-oxide, or any combination thereof.

In another embodiment, the present invention provides a method of suppressing or inhibiting androgen-deprivation induced osteoporosis in a male human subject suffering from prostate cancer, comprising the step of administering to said subject 80 mg per day of Toremifene and/or its analog, derivative, isomer, metabolite, pharmaceutically acceptable salt, pharmaceutical product, hydrate, N-oxide, or any combination thereof.

In another embodiment, the present invention provides a method of reducing the risk of developing androgen-deprivation induced osteoporosis in a male human subject suffering from prostate cancer, comprising the step of administering to said subject 80 mg per day of Toremifene and/or its analog, derivative, isomer, metabolite, pharmaceutically acceptable salt, pharmaceutical product, hydrate, N-oxide, or any combination thereof.

In another embodiment, the present invention provides a method of treating androgen-deprivation induced loss of BMD in a male subject suffering from prostate cancer, comprising the step of administering to said subject 80 mg per day of Toremifene and/or its analog, derivative, isomer, metabolite, pharmaceutically acceptable salt, pharmaceutical product, hydrate, N-oxide, or any combination thereof.

In another embodiment, the present invention provides a method of preventing androgen-deprivation induced loss of BMD in a male subject suffering from prostate cancer, comprising the step of administering to said subject 80 mg per day of Toremifene and/or its analog, derivative, isomer, metabolite, pharmaceutically acceptable salt, pharmaceutical product, hydrate, N-oxide, or any combination thereof.

In another embodiment, the present invention provides a method of suppressing or inhibiting androgen-deprivation induced loss of BMD in a male subject suffering from prostate cancer, comprising the step of administering to said subject 80 mg per day of Toremifene and/or its analog, derivative, isomer, metabolite, pharmaceutically acceptable salt, pharmaceutical product, hydrate, N-oxide, or any combination thereof.

In another embodiment, the present invention provides a method of reducing the risk of developing androgen-deprivation induced loss of BMD in a male subject suffering from prostate cancer comprising the step of administering to said subject 80 mg per day of Toremifene and/or its analog, derivative, isomer, metabolite, pharmaceutically acceptable salt, pharmaceutical product, hydrate, N-oxide, or any combination thereof.

In another embodiment, the present invention provides a method of treating androgen-deprivation induced bone fractures in a male subject suffering from prostate cancer, comprising the step of administering to said subject 80 mg per day of Toremifene and/or its analog, derivative, isomer, metabolite, pharmaceutically acceptable salt, pharmaceutical product, hydrate, N-oxide, or any combination thereof.

In another embodiment, the present invention provides a method of preventing androgen-deprivation induced bone fractures in a male subject suffering from prostate cancer, comprising the step of administering to said subject 80 mg per day of Toremifene and/or its analog, derivative, isomer, metabolite, pharmaceutically acceptable salt, pharmaceutical product, hydrate, N-oxide, or any combination thereof.

In another embodiment, the present invention provides a method of suppressing or inhibiting androgen-deprivation induced bone fractures in a male subject suffering from prostate cancer, comprising the step of administering to said subject 80 mg per day of Toremifene and/or its analog, derivative, isomer, metabolite, pharmaceutically acceptable salt, pharmaceutical product, hydrate, N-oxide, or any combination thereof.

In another embodiment, the present invention provides a method of reducing the risk of developing androgen-deprivation induced bone fractures in a male subject suffering from prostate cancer, comprising the step of administering to said subject 80 mg per day of Toremifene and/or its analog, derivative, isomer, metabolite, pharmaceutically acceptable salt, pharmaceutical product, hydrate, N-oxide, or any combination thereof.

Osteoporosis is a systemic skeletal disease, characterized by low bone mass and deterioration of bone tissue, with a consequent increase in bone fragility and susceptibility to fracture. In osteoporotic patients, bone strength is abnormal, with a resulting increase in the risk of fracture. Osteoporosis depletes both the calcium and the protein collagen normally found in the bone, resulting in either abnormal bone quality or decreased bone density. Bones that are affected by osteoporosis can fracture with only a minor fall or injury that normally would not cause a bone fracture. The fracture can be either in the form of cracking (as in a hip fracture) or collapsing (as in a compression fracture of the spine). The spine, hips, and wrists are common areas of osteoporosis bone fractures, although fractures can also occur in other skeletal areas.

BMD is a measured calculation of the true mass of bone. The absolute amount of bone as measured by bone mineral density (BMD) generally correlates with bone strength and its ability to bear weight. By measuring BMD, it is possible to predict fracture risk in the same manner that measuring blood pressure can help predict the risk of stroke.

BMD in one embodiment can be measured by known bone-mineral content mapping techniques. Bone density of the hip, spine, wrist, or calcaneus may be measured by a variety of techniques. The preferred method of BMD measurement is dual-energy x-ray densitometry (DXA). BMD of the hip, antero-posterior (AP) spine, lateral spine, and wrist can be measured using this technology. Measurement at any site predicts overall risk of fracture, but information from a specific site is the best predictor of fracture at that site. Quantitative computerized tomography (QCT) is also used to measure BMD of the spine. See for example, “Nuclear Medicine: “Quantitative Procedures”. by Wahner H W, Dunn W L, Thorsen H C, et al, published by Toronto Little, Brown & Co., 1983, (see pages 107-132). An article entitled “Assessment of Bone Mineral Part 1” appeared in the Journal of Nuclear Medicine, pp 1134-1141, (1984). Another article entitled “Bone Mineral Density of The Radius” appeared in Vol. 26, No. 11, (1985) Nov. Journal of Nuclear Medicine at pp 13-39. Abstracts on the use of gamma cameras for bone-mineral content measurements are (a) S. Hoory et al, Radiology, Vol. 157(P), p. 87 (1985), and (b) C. R. Wilson et al, Radiology, Vol. 157(P), p. 88 (1985).

The present invention provides a safe and effective method for treating, preventing, suppressing, inhibiting or reducing the risk of developing androgen-deprivation induced osteoporosis and/or loss of BMD and is particularly useful for treating male subjects suffering from prostate cancer having an elevated risk of developing androgen-deprivation induced osteoporosis.

Toremifene, at the doses described herein is effective at treating, suppressing or inhibiting osteopenia accompanied by bone loss. “Osteopenia” refers to decreased calcification or density of bone. This is a term which encompasses all skeletal systems in which such a condition is noted.

The invention includes the administration of “pharmaceutically acceptable salts” of Toremifene. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

Pharmaceutical Compositions

In one embodiment, the methods of the present invention comprise administering a pharmaceutical composition comprising Toremifene at a dosage which results in the delivery of 80 mg to the subject, in single dose units. The pharmaceutical composition is administered to a male human subject suffering from prostate cancer; for treating and/or preventing androgen-deprivation induced osteoporosis and/or loss of BMD; for suppressing or inhibiting androgen-deprivation induced osteoporosis and/or loss of BMD; and/or for reducing the risk of developing androgen-deprivation induced osteoporosis and/or loss of BMD in the male subject.

As used herein, “pharmaceutical composition” means a “therapeutically effective amount” of the active ingredient, i.e. Toremifene, together with a pharmaceutically acceptable carrier or diluent. A “therapeutically effective amount” as used herein refers to that amount which provides a therapeutic effect for a given condition and administration regimen.

The pharmaceutical compositions containing Toremifene can be administered to a subject by any method known to a person skilled in the art, such as parenterally, paracancerally, transmucosally, transdermally, intramuscularly, intravenously, intradermally, subcutaneously, intraperitonealy, intraventricularly, intracranially, intravaginally or intratumorally.

In one embodiment, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In one embodiment of the present invention, Toremifene is formulated in a capsule. In accordance with this embodiment, the compositions of the present invention comprise, in addition to Toremifene and the inert carrier or diluent, a hard gelating capsule.

Further, in another embodiment, the pharmaceutical compositions are administered by intravenous, intraarterial, or intramuscular injection of a liquid preparation. Suitable liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intraarterially, and are thus formulated in a form suitable for intraarterial administration. In another embodiment, the pharmaceutical compositions are administered intramuscularly, and are thus formulated in a form suitable for intramuscular administration.

Further, in another embodiment, the pharmaceutical compositions are administered topically to body surfaces, and are thus formulated in a form suitable for topical administration. Suitable topical formulations include gels, ointments, creams, lotions, drops and the like. For topical administration, Toremifene is formulated in a composition comprising a physiologically acceptable diluent with or without a pharmaceutical carrier.

Further, in another embodiment, the pharmaceutical compositions are administered as a suppository, for example a rectal suppository or a urethral suppository. Further, in another embodiment, the pharmaceutical compositions are administered by subcutaneous implantation of a pellet. In a further embodiment, the pellet provides for controlled release of Toremifene over a period of time.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

As used herein “pharmaceutically acceptable carriers or diluents” are well known to those skilled in the art. The carrier or diluent may be a solid carrier or diluent for solid formulations, a liquid carrier or diluent for liquid formulations, or mixtures thereof.

Solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

In one embodiment, the pharmaceutical compositions provided herein are controlled-release compositions, i.e. compositions in which Toremifene is released over a period of time after administration. Controlled- or sustained-release compositions include formulation in lipophilic depots (e.g. fatty acids, waxes, oils). In another embodiment, the composition is an immediate-release composition, i.e. a composition in which Toremifene is released immediately after administration.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled-release systems are discussed in the review by Langer (Science 249:1527-1533 (1990).

The compositions may also include incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention is the modification of Toremifene by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The preparation of pharmaceutical compositions which contain an active component is well understood in the art, for example by mixing, granulating, or tablet-forming processes. The active therapeutic ingredient is often mixed with excipients which are pharmaceutically acceptable and compatible with the active ingredient. For oral administration, Toremifene is mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, Toremifene is converted into a solution, suspension, or emulsion, if desired with the substances customary and suitable for this purpose, for example, solubilizers or other.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicine, the salts of Toremifene are pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

As defined herein, “contacting” means that Toremifene is introduced into a sample containing the enzyme in a test tube, flask, tissue culture, chip, array, plate, microplate, capillary, or the like, and incubated at a temperature and time sufficient to permit binding of the anti-estrogen to the enzyme. Methods for contacting the samples with Toremifene or other specific binding components are known to those skilled in the art and may be selected depending on the type of assay protocol to be run. Incubation methods are also standard and are known to those skilled in the art.

In another embodiment, the term “contacting” means that Toremifene is introduced into a subject receiving treatment, and Toremifene is allowed to come in contact with the androgen receptor in vivo.

As used herein, the term “treating” includes preventative as well as disorder remitative treatment. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission.

As used herein, the term “administering” refers to bringing a subject in contact with an anti-estrogen compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

In one embodiment, the methods of the present invention comprise administering Toremifene as the sole active ingredient. However, also encompassed within the scope of the present invention are methods for hormone therapy, for treating prostate cancer, for delaying the progression of prostate cancer, and for preventing and/or treating the recurrence of prostate cancer, which comprise administering Toremifene at a dose of about 80 mg per day, in combination with one or more therapeutic agents. These agents include, but are not limited to: LHRH analogs, reversible antiandrogens (such as bicalutamide or flutamide), additional anti-estrogens, anticancer drugs, 5-alpha reductase inhibitors, aromatase inhibitors, progestins, selective androgen receptor modulators (SARMS) or agents acting through other nuclear hormone receptors.

Thus, in one embodiment, the methods of the present invention include using compositions and pharmaceutical compositions providing Toremifene at a dose of 80 mg per day and further comprising an LHRH analog. In another embodiment, the methods of the present invention include using compositions and pharmaceutical compositions providing Toremifene at a dose of 80 mg per day and further comprising a reversible antiandrogen. In another embodiment, the methods of the present invention include using compositions and pharmaceutical compositions providing Toremifene at a dose of 80 mg per day and further comprising an anti-estrogen. In another embodiment, the methods of the present invention include using compositions and pharmaceutical compositions providing Toremifene at a dose of 80 mg per day and further comprising with an anticancer drug. In another embodiment, the methods of the present invention include using compositions and pharmaceutical compositions providing Toremifene at a dose of 80 mg per day and further comprising a 5-alpha reductase inhibitor. In another embodiment, the methods of the present invention include using compositions and pharmaceutical compositions providing Toremifene at a dose of 80 mg per day and further comprising an aromatase inhibitor. In another embodiment, the methods of the present invention include using compositions and pharmaceutical compositions providing Toremifene at a dose of 80 mg per day and further comprising a progestin. In another embodiment, the methods of the present invention include using compositions and pharmaceutical compositions comprising providing Toremifene at a dose of 80 mg per day and further comprising a SARM. In another embodiment, the methods of the present invention include using compositions and pharmaceutical compositions providing Toremifene at a dose of 80 mg per day and further comprising an agent acting through other nuclear hormone receptors.

The following examples are presented in order to more fully illustrate the preferred embodiments of the invention. They should in no way, however, be construed as limiting the broad scope of the invention.

### EXPERIMENTAL DETAILS SECTION

### Example 1

### Effect of Toremifene on Bone Turnover in Human Male Subjects

In a Phase IIa clinical trial to determine whether Toremifene has chemopreventive activity against prostate cancer, 18 men with high-grade prostatic intraepithelial neoplasia (HGPIN) were treated with 60 mg/d of Toremifene for 4 months. At Day 120 there was a significant reduction from baseline in serum calcium (mean −0.12, p=0.005) and at both day 60 and day 120, alkaline phosphatase was significantly decreased compared to baseline (mean=−18.7 at Day 60 and −21.0 at Day 120, and p<0.001 for both visits).

These clinical data demonstrate that the anti-estrogen Toremifene showed estrogenic effects on bone favorably affecting bone turnover markers in men.

### Example 2

### Effect of 80 mg Toremifene on Increasing Bone Density in a Human Clinical Trial

Men with a histologically confirmed diagnosis of prostate cancer who have been treated with ADT for at least 6 months, greater than 70 years of age or at least 50 years of age with evidence of osteopenia by baseline dual energy X-ray absorptiometry (DEXA) scan were assigned randomly to receive either toremifene citrate 80 mg daily or placebo. Treatment was continued for 12 months at which time a DEXA scan was performed.

200 men were assessed in this study.

Table 2-1 shows the age distribution of the subjects in the study.

| | | Toremifene, | |
|---|---|---|---|
| Variable | Placebo | 80 mg | Total |
| Sample size | 104 | 93 | 197 |
| Mean | 77.5 | 76.3 | 76.9 |
| SD | 6.45 | 6.89 | 6.67 |
| Median | 79.0 | 77.0 | 78.0 |
| Minimum | 60 | 54 | 54 |
| Maximum | 90 | 89 | 90 |

Table 2-2 demonstrates mean change from baseline to month 12 in lumbar bone mineral density for subjects that have completed 12 months of treatment

| | | | | | p-value |
|---|---|---|---|---|---|
| | | | Toremifene | | vs. |
| | Visit | Placebo | 80 mg | Pooled | Placebo |
| | Statistic | (n = 104) | (n = 93) | SD | [1] |
| Baseline | n | 104 | 93 | | |
| | Mean | 1.0388 | 1.1025 | | |
| | Median | 1.0075 | 1.0880 | | |
| | LS Mean | 1.0388 | 1.1025 | 0.20806 | 0.033* |
| | SE LS Mean | 0.02040 | 0.02157 | | |
| | Min-Max | 0.694-1.670 | 0.692-1.761 | | |
| Change | n | 104 | 93 | | |
| from | | | | | |
| Baseline to | | | | | |
| Month 12 | | | | | |
| | Mean | −0.0076 | 0.0164 | | |
| | Median | −0.0050 | 0.0100 | | |
| | LS Mean | −0.0080 | 0.0169 | 0.03492 | <0.001* |
| | SE LS Mean | 0.00344 | 0.00364 | | |
| | Min-Max | −0.122-0.060 | −0.050-0.117 | | |

*Denotes statistical significance at the 0.045 level. [1]P-values are from an ANOVA with treatment as the factor at the Baseline Visit and from an ANOVA with treatment and baseline BMD as the factors at the Month 12 Visit.

Table 2-3 shows mean change from baseline to month 12 in the lumbar bone mineral density for subjects that have completed 12 months of treatment, including the site.

| | | | Toremifene | | |
|---|---|---|---|---|---|
| | Visit | Placebo | 80 mg | | p-value vs. |
| | Statistic | (n = 104) | (n = 93) | Pooled SD | Placebo [1] |
| Baseline | n | 104 | 93 | | |
| | Mean | 1.0388 | 1.1025 | | |
| | Median | 1.0075 | 1.0880 | | |
| | LS Mean | 1.0500 | 1.0972 | 0.18527 | 0.099 |
| | SE LS Mean | 0.02125 | 0.02367 | | |
| | Min-Max | 0.694-1.670 | 0.692-1.761 | | |
| Change | n | 104 | 93 | | |
| from | | | | | |
| Baseline to | | | | | |
| Month 12 | | | | | |
| | Mean | 0.0076 | 0.0164 | | |
| | Median | 0.0050 | 0.0100 | | |
| | LS Mean | 0.0044 | 0.0193 | 0.03452 | <0.001* |
| | SE LS Mean | 0.00397 | 0.00443 | | |
| | Min-Max | −0.122-0.060 | −0.050-0.117 | | |

| | Visit | | Toremifene | Pooled |
|---|---|---|---|---|
| | Statistic | Placebo | 80 mg | SD |
| Baseline | n | 104 | 93 | |
| | Mean | 1.039 | 1.102 | 0.2100 |
| | SD | 0.1935 | 0.2233 | |
| | Median | 1.008 | 1.088 | |
| | LS Mean | 1.0500 | 1.0972 | 0.18527 |
| | Min-Max | 0.69-1.670 | 0.69-1.76 | |
| Change | n | 104 | 93 | |
| from | | | | |
| Baseline | | | | |
| to Month | | | | |
| 12 | | | | |
| | Mean | −0.694 | 1.593 | |
| | SD | 3.2742 | 3.4025 | |
| | Median | −0.4555 | 0.960 | |
| | Min-Max | −12.13-6.51 | −5.02-9.80 | |

Table 2-4 summarizes the percent change from baseline in lumbar bone mineral density, for subjects who have completed 12 months of treatment.

Table 2-5 summarizes mean changes from baseline to month 12 in femur bone mineral density.

| | | | Toremifene | Pooled | p-value vs. |
|---|---|---|---|---|---|
| | Statistic | Placebo | 80 mg | SD | Placebo [1] |
| Baseline | n | 103 | 92 | | |
| | Mean | 0.7636 | 0.8033 | | |
| | Median | 0.7570 | 0.7760 | | |
| | LS Mean | 0.7636 | 0.8033 | 0.15258 | 0.071 |
| | SE LS Mean | 0.01503 | 0.01591 | | |
| | Min-Max | 0.515-1.114 | 0.528-1.198 | | |
| Changes | n | 103 | 92 | | |
| from | Mean | −0.0103 | 0.0013 | | |
| Baseline | Median | −0.0090 | 0.0030 | | |
| at | LS Mean | −0.0105 | 0.0016 | 0.03145 | 0.009* |
| month | SE LS Mean | 0.00311 | 0.00329 | | |
| 12 | Min-Max | −0.120-0.064 | −0.132-0.077 | | |

Table 2-6 summarizes mean changes from baseline to month 12 in femur bone mineral density, including the site.

| | | | Toremifene | Pooled | p-value vs. |
|---|---|---|---|---|---|
| | Statistic | Placebo | 80 mg | SD | Placebo [1] |
| Baseline | n | 103 | 92 | | |
| | Mean | 0.7636 | 0.8033 | | |
| | Median | 0.7570 | 0.7760 | | |
| | LS Mean | 0.7756 | 0.7833 | 0.13216 | 0.710 |
| | SE LS Mean | 0.01539 | 0.01708 | | |
| | Min-Max | 0.515-1.114 | 0.528-1.198 | | |
| Change | n | 103 | 92 | | |
| from | Mean | −0.0103 | 0.0013 | | |
| Baseline to | Median | −0.0090 | 0.0030 | | |
| Month 12 | LS Mean | −0.0125 | 0.0014 | 0.03168 | 0.006* |
| | SE LS Mean | 0.00369 | 0.00409 | | |
| | Min-Max | −0.120-0.064 | −0.132-0.077 | | |

Table 2-7 shows the percentage change from baseline to month 12 in femur bone mineral density.

| | | | | Pooled |
|---|---|---|---|---|
| | Statistic | Placebo | Toremifene 80 mg | SD |
| Baseline | n | 103 | 92 | |
| | Mean | 0.764 | 0.803 | 0.1535 |
| | SD | 0.1346 | 0.1705 | |
| | Median | 0.757 | 0.776 | |
| | Min-Max | 0.52-1.11 | 0.53-1.20 | |
| Percentage | n | 103 | 92 | |
| Change | Mean | −1.302 | 0.173 | |
| from | SD | 4.2801 | 3.6716 | 4.0621 |
| Baseline to | Median | −1.190 | 0.505 | |
| Month 12 | Min-Max | −18.13-8.38 | −13.02-10.91 | |

Table 2-8 shows the mean change from baseline to month 12 in the hip bone mineral density for subjects that have completed 12 months of treatment.

| | | | Toremifene | Pooled | p-value vs. |
|---|---|---|---|---|---|
| | Statistic | Placebo | 80 mg | SD | Placebo [1] |
| Baseline | n | 103 | 91 | | |
| | Mean | 0.8917 | 0.9181 | | |
| | Median | 0.8900 | 0.8950 | | |
| | LS Mean | 0.8917 | 0.9181 | 0.15248 | 0.231 |
| | SE LS Mean | 0.01502 | 0.01598 | | |
| | Min-Max | 0.602-1.257 | 0.608-1.331 | | |
| Change | n | 103 | 91 | | |
| from | Mean | −0.0112 | 0.0049 | | |
| Baseline to | Median | −0.0100 | 0.0040 | | |
| Month 12 | LS Mean | −0.0113 | 0.0050 | 0.03313 | |
| | SE LS Mean | 0.00327 | 0.00348 | | 0.001* |
| | Min-Max | −0.131-0.105 | −0.083-0.290 | | |

Table 2-9 shows mean change from baseline to month 12 in the hip bone mineral density, including the site.

| | | | Toremifene | Pooled | p-value vs. |
|---|---|---|---|---|---|
| | Statistic | Placebo | 80 mg | SD | Placebo [1] |
| Baseline | n | 103 | 91 | | |
| | Mean | 0.8917 | 0.9181 | | |
| | Median | 0.8900 | 0.8950 | | |
| | LS Mean | 0.8914 | 0.8934 | 0.14733 | 0.931 |
| | SE LS Mean | 0.01716 | 0.01914 | | |
| | Min-Max | 0.602-1.257 | 0.608-1.331 | | |
| Change | n | 103 | 91 | | |
| from | Mean | −0.0112 | 0.0049 | | |
| Baseline to | Median | −0.0100 | 0.0040 | | |
| Month 12 | LS Mean | −0.0137 | 0.0049 | 0.03459 | 0.001* |
| | SE LS Mean | 0.00404 | 0.00450 | | |
| | Min-Max | −0.131-0.105 | −0.083-0.290 | | |

Table 2-10 shows the percentage change from baseline to month 12 in hip bone mineral density, including the site.

| | | | Toremifene | Pooled |
|---|---|---|---|---|
| | Statistic | Placebo | 80 mg | SD |
| Baseline | n | 103 | 91 | |
| | Mean | 0.892 | 0.918 | 0.1527 |
| | SD | 0.1364 | 0.1688 | |
| | Median | 0.890 | 0.895 | |
| | Min-Max | 0.60-1.26 | 0.61-1.33 | |
| Percentage | n | 103 | 91 | |
| Change | Mean | −1.284 | 0.672 | 4.4508 |
| from | SD | 3.0961 | 5.4371 | |
| Baseline to | Median | −1.210 | 0.420 | |
| Month 12 | Min-Max | −12.93-8.35 | −6.65-47.31 | |

Cumulative DEXA results compared to baseline were as summarized in table 4-11 below:

Cumulative DEXA results compared to baseline were
as summarized in table 4-11 below:

| | Toremifene | | | |
|---|---|---|---|---|
| | 80 mg (% | Placebo (% | | |
| | change from | change from | Treatment | |
| | baseline) | baseline) | Effect (%) | p value |
| Lumbar | 1.6 | −0.69 | 2.3 | <0.001 |
| Spine | | | | |
| Total Hip | 0.67 | −1.3 | 2.0 | 0.001 |
| Femoral | 0.17 | −1.3 | 1.5 | 0.009 |
| Neck | | | | |

Toremifene citrate produced statistically significant and clinically meaningful changes in bone mineral density in men treated with ADT for prostate cancer. A clinically meaningful decrease in BMD was apparent in the placebo group confirming the occurrence of accelerated BMD loss in men treated with ADT. The magnitude of BMD preservation and increase seen in men treated with toremifene citrate was similar to that seen in clinical trials with SERMs, in treating reductions in fracture rates in post-menopausal women. Toremifene citrate thus will provide a fracture reduction benefit.

It will be appreciated by a person skilled in the art that the present invention is not limited by what has been particularly shown and described hereinabove. Rather, the scope of the invention is defined by the claims which follow:

What is claimed is:

1. A method of treating androgen-deprivation induced osteoporosis in a male human subject suffering from prostate cancer, said method comprising the step of administering 80 mg per day of Toremifene, or a pharmaceutically acceptable salt thereof to said subject.

2. The method according to claim 1, wherein said administering comprises administering a pharmaceutical composition comprising said Toremifene and/or its analog, derivative, isomer, metabolite, pharmaceutically acceptable salt, pharmaceutical product, hydrate, N-oxide, or any combination thereof; and a pharmaceutically acceptable carrier.

3. The method according to claim 2, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

4. The method according to claim 2 wherein said pharmaceutical composition is a pellet, a tablet, a capsule, a solution, a suspension, an emulsion, an elixir, a gel, a cream, a suppository or a parenteral formulation.

5. A method of preventing androgen-deprivation induced osteoporosis in a male human subject suffering from prostate cancer, said method comprising the step of administering 80 mg per day Toremifene, or a pharmaceutically acceptable salt thereof to said subject.

6. The method according to claim 5, wherein said administering comprises administering a pharmaceutical composition comprising said Toremifene and/or its analog, derivative, isomer, metabolite, pharmaceutically acceptable salt, pharmaceutical product, hydrate, N-oxide, or any combination thereof; and a pharmaceutically acceptable carrier.

7. The method according to claim 6, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

8. The method according to claim 6 wherein said pharmaceutical composition is a pellet, a tablet, a capsule, a solution, a suspension, an emulsion, an elixir, a gel, a cream, a suppository or a parenteral formulation.

9. A method of suppressing, inhibiting or reducing the risk of developing androgen-deprivation induced osteoporosis in a male human subject suffering from prostate cancer, said method comprising the step of administering 80 mg per day of Toremifene, or a pharmaceutically acceptable salt thereof to said subject.

10. The method according to claim 9, wherein said administering comprises administering a pharmaceutical composition comprising said Toremifene and/or its analog, derivative, isomer, metabolite, pharmaceutically acceptable salt, pharmaceutical product, hydrate, N-oxide, or any combination thereof; and a pharmaceutically acceptable carrier.

11. The method according to claim 10, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

12. The method according to claim 11 wherein said pharmaceutical composition is a pellet, a tablet, a capsule, a solution, a suspension, an emulsion, an elixir, a gel, a cream, a suppository or a parenteral formulation.

13. A method of treating androgen-deprivation induced loss of bone mineral density (BMD) in a male human subject suffering from prostate cancer, said method comprising the step of administering 80 mg per day Toremifene, or a pharmaceutically acceptable salt thereof to said subject.

14. The method according to claim 13, wherein said administering comprises administering a pharmaceutical composition comprising said Toremifene and/or its analog, derivative, isomer, metabolite, pharmaceutically acceptable salt, pharmaceutical product, hydrate, N-oxide, or any combination thereof; and a pharmaceutically acceptable carrier.

15. The method according to claim 13, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

16. The method according to claim 13 wherein said pharmaceutical composition is a pellet, a tablet, a capsule, a solution, a suspension, an emulsion, an elixir, a gel, a cream, a suppository or a parenteral formulation.

17. A method of preventing androgen-deprivation induced loss of bone mineral density (BMD) in a male human subject suffering from prostate cancer, said method comprising the step of administering 80 mg per day of Toremifene, or a pharmaceutically acceptable salt thereof to said subject.

18. The method according to claim 17, wherein said administering comprises administering a pharmaceutical composition comprising said Toremifene and/or its analog, derivative, isomer, metabolite, pharmaceutically acceptable salt, pharmaceutical product, hydrate, N-oxide, or any combination thereof; and a pharmaceutically acceptable carrier.

19. The method according to claim 17, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

20. The method according to claim 17 wherein said pharmaceutical composition is a pellet, a tablet, a capsule, a solution, a suspension, an emulsion, an elixir, a gel, a cream, a suppository or a parenteral formulation.

21. A method of suppressing, inhibiting or reducing the risk of developing androgen-deprivation induced loss of bone mineral density (BMD) in a male human subject suffering from prostate cancer, said method comprising the step of administering 80 mg per day of Toremifene, or a pharmaceutically acceptable salt thereof to said subject.

22. The method according to claim 21, wherein said administering comprises administering a pharmaceutical composition comprising said Toremifene and/or its analog, derivative, isomer, metabolite, pharmaceutically acceptable salt, pharmaceutical product, hydrate, N-oxide, or any combination thereof; and a pharmaceutically acceptable carrier.

23. The method according to claim 21, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

24. The method according to claim 21 wherein said pharmaceutical composition is a pellet, a tablet, a capsule, a solution, a suspension, an emulsion, an elixir, a gel, a cream, a suppository or a parenteral formulation.

25. A method of treating androgen-deprivation induced bone fractures in a male human subject suffering from prostate cancer, said method comprising the step of administering Toremifene, or a pharmaceutically acceptable salt thereof to said subject.

26. The method according to claim 25, wherein said administering comprises administering a pharmaceutical composition comprising said Toremifene and/or its analog, derivative, isomer, metabolite, pharmaceutically acceptable salt, pharmaceutical product, hydrate, N-oxide, or any combination thereof; and a pharmaceutically acceptable carrier.

27. The method according to claim 26, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

28. The method according to claim 26 wherein said pharmaceutical composition is a pellet, a tablet, a capsule, a solution, a suspension, an emulsion, an elixir, a gel, a cream, a suppository or a parenteral formulation.

29. A method of preventing androgen-deprivation induced bone fractures in a male human subject suffering from prostate cancer, said method comprising the step of administering 80 mg/kg per day of Toremifene, or a pharmaceutically acceptable salt thereof to said subject.

30. The method according to claim 29, wherein said administering comprises administering a pharmaceutical composition comprising said Toremifene and/or its analog, derivative, isomer, metabolite, pharmaceutically acceptable salt, pharmaceutical product, hydrate, N-oxide, or any combination thereof; and a pharmaceutically acceptable carrier.

31. The method according to claim 30, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

32. The method according to claim 30 wherein said pharmaceutical composition is a pellet, a tablet, a capsule, a solution, a suspension, an emulsion, an elixir, a gel, a cream, a suppository or a parenteral formulation.

33. A method of suppressing, inhibiting or reducing the risk of developing androgen-deprivation induced bone fractures in a male human subject suffering from prostate cancer, said method comprising the step of administering 80 mg/day of Toremifene, or a pharmaceutically acceptable salt thereof to said subject.

34. The method according to claim 33, wherein said administering comprises administering a pharmaceutical composition comprising said anti-estrogen and/or its analog, derivative, isomer, metabolite, pharmaceutically acceptable salt, pharmaceutical product, hydrate, N-oxide, or any combination thereof; and a pharmaceutically acceptable carrier.

35. The method according to claim 33, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

36. The method according to claim 33 wherein said pharmaceutical composition is a pellet, a tablet, a capsule, a solution, a suspension, an emulsion, an elixir, a gel, a cream, a suppository or a parenteral formulation.
